(19) United States
(12) Patent Application Publication
(10) Pub. No.: US 2024/0252797 A1
(43) Pub. Date: Aug. 1, 2024

(54) MULTILAYER MICRONEEDLE ARRAY AND METHOD FOR MANUFACTURING SAME

(71) Applicant: DERMAJECT CO., LTD., Uiwang-si Gyeonggi-do (KR)

(72) Inventors: Kyung-Rae BAEK, Yongin-si Gyeonggi-do (KR); Yong-Hyun JUNG, Pyeongtaek-si Gyeonggi-do (KR); Jae-Min CHOI, Uiwang-si Gyeonggi-do (KR)

(21) Appl. No.: 18/564,051

(22) PCT Filed: Jul. 2, 2021
(86) PCT No.: PCT/KR2021/008414

§ 371 Date: Nov. 24, 2023

(30) Foreign Application Priority Data
May 24, 2021 (KR) 10-2021-0066182

(51) Int. Cl. A61M 37/00; A61K 9/00

(52) U.S. Cl. CPC A61M 37/0015; A61K 9/0014; A61K 9/0021; A61M 2037/0023; A61M 2037/0053; A61M 2205/0244

(57) ABSTRACT

The present invention relates to a microneedle comprising a drug part, a separational function part which is water-soluble, and a basic part comprising a photocurable resin. Also, the present invention relates to a microneedle array comprising the microneedle according to the present invention. In addition, the present invention relates to a method for manufacturing a microneedle array, the method comprising: a step for placing a drug part composition in a mold and drying a solvent to form a drug part; a step for placing a separational function part composition on the drug part and dying the same to form a separational function part; and a step for applying a base part composition on the separational function part and curing the base part composition.

### CROSS-REFERENCE TO RELATED APPLICATION

The present application is a national stage filing under 35 U.S.C § 371 of PCT application number PCT/KR2021/008414 filed on Jul. 2, 2021 which is based upon and claims the benefit of priorities to Korean Patent Application No. 10-2021-0066182 filed on May 24 2021 in the Korean Intellectual Property Office. All of the aforementioned applications are hereby incorporated by reference in their entireties.

### TECHNICAL FIELD

The present disclosure relates to a multilayer microneedle array and a method for manufacturing the same.

### BACKGROUND ART

Microneedles is developed to deliver a drug efficiently across the skin barrier, and ensures convenience of a transdermal patch as well as enabling a direct transdermal delivery of a drug, reducing resistance to an existing syringe, a pain, and a risk of infection. For example, a microneedle is disclosed in KR Patent Publication No. 10-2015-0082234, KR Patent No. 10-2036921, KR Patent No. 10-1776659 and the like, and a manufacturing method of an ocular microneedle syringe is disclosed in KR Patent No. 10-2060138.

Microneedles are manufactured such as a hollow microneedle with reduced size of conventional syringe, a coated microneedle where a drug is coated on a microneedle made of metal or plastic, a microneedle where a biocompatible polymer and a drug are mixed, and the like, in various ways, to deliver a drug into the skin efficiently.

Among the microneedles, a biodegradable microneedle or a soluble microneedle made of a biocompatible material having biodegradability or solubility do not ensure a fast delivery speed of a drug, and cause a difference in an amount of a delivered drug. Additionally, since the biodegradable microneedle or the soluble microneedle are dissolved or biodegradable in the body, after they are attached to the skin, a certain period of time is required for a mounted drug to be delivered completely, and some of them can be removed together in an undissolved state. Since a drug is included in a tip part and a base part, in most soluble or biodegradable microneedles, it is likely to cause a loss of an expensive drug, in the case of an expensive drug. To prevent this from happening, concentration of a drug on the tip part, and delivery of an accurate dose of a drug need to be taken into account, in manufacturing a microneedle. Additionally, since secondary infection is likely to occur through a gap between the skin and the needle, causing a foreign body sensation or a pain, while the microneedle is attached to the skin, there is a growing demand for a needle in which a base part and a tip part separate rapidly after use of the microneedle and which enhances user safety and user convenience.

However, existing double layer microneedles in KR Patent No. 10-2036921 and KR Patent No. 10-1776659 and the like are manufactured in such a way that microneedles without a tip part are manufactured with a thermoplastic resin in advance, and coupled to a tip part later at a time of manufacturing the microneedles, causing high costs and low production efficiency. Additionally, in relation to a method of separating the tip part, in the case where the tip part is separated based on a difference between a force of holding the tip part by the skin and a force of coupling the tip part and a guider part, the guider part and the tip part are likely to be removed together, if the tip part and the guider part are not separated properly. Further, regarding KR Patent No. 10-2060138, in the case of a microneedle syringe comprised of one microneedle per injector, an amount of a mounted dung is limited to a level of ng, and to embody a microarray for increasing an amount of a mounted drug, a plurality of injectors needs to be provided, making it hard to mass-produce the microarray.

### DESCRIPTION OF THE INVENTION

### Technical Problems

The objective of the present disclosure is to provide a multilayer microneedle array and a method for manufacturing the same enabling an injection of an accurate dose of a drug to the skin.

### Technical Solutions

To achieve the above objective, according to the present disclosure, provided is
a microneedle comprising a drug part, a separational function part that is water-soluble, and a base part that comprises a photocurable resin.

Additionally, according to the present disclosure, provided is a microneedle array comprising the microneedle of the present disclosure.

Further, according to the present disclosure, provided is a method for manufacturing a microneedle array comprising,
placing a drug part composition in a mold and drying a solvent, to form a drug part,
placing a separational function part composition on the drug part and drying the same, to form a separational function part, and
applying a base part composition on the separational function part and curing the same.

### Advantageous Effects

A microneedle array of the present disclosure enables an injection of an accurate dose of a drug into the skin, and separates within a short period of time, based on dissolution of a separational function part comprised of a water-soluble polymer, making it possible to remove the microneedle array within a short period of time and suppress a portion of a base part from remaining in the skin, with no need to attach the microneedle array to the skin for a long period of time.

### BRIEF DESCRIPTION OF THE DRAWINGS

FIG. 1a is a view showing the structure of one microneedle, in the microneedle array of the present disclosure. FIG. 1b is a view showing embodiments of the microneedle of the present disclosure.

FIG. 2 is a view showing the microneedle array of the present disclosure.

FIG. 3 is a view showing that a microneedle array is attached to the skin and then a base part is separated.

FIG. 4a is a view showing a problem of an existing double layer microneedle array. That is, FIG. 4a is a view showing a structure prior to attachment of an existing double layer microneedle array to the skin, and the structure of a portion remaining at the microneedle array after the attachment.

FIG. 4b is a view showing a structure prior to attachment of a multilayer microneedle array of the present disclosure to the skin, and the structure of a portion remaining at the microneedle array after the attachment.

FIG. 5 is a schematic view showing a method for manufacturing the multilayered microneedle array of the present disclosure, with a portion of one microneedle.

FIG. 6a is a micrograph showing a microneedle array of embodiment 1. FIG. 6b is a micrograph showing a microneedle array of embodiment 2. FIG. 6c is a micrograph showing a microneedle array of comparative example 1. FIG. 6d is a micrograph showing a microneedle array of comparative example 2.

FIG. 7a is a view showing a base part after attachment of the microneedle array of embodiment 1 to the skin of a pig and then removal of the microneedle array from the skin of the pig. FIGS. 7b and 7c are views showing an attached surface of the skin of a pig after attachment of the microneedle array of embodiment 1 to the skin of the pig and then removal of the microneedle array from the skin of the pig.

FIG. 8a is a view showing a base part after attachment of the microneedle array of embodiment 2 to the skin of a pig and then removal of the microneedle array from the skin of the pig. FIG. 8b is a view showing an attached surface of the skin of a pig after attachment of the microneedle array of embodiment 2 to the skin of the pig and then removal of the microneedle array from the skin of the pig.

FIG. 9a is a view showing a base part after attachment of the microneedle array of comparative example 1 to the skin of a pig and then removal of the microneedle array from the skin of the pig. FIG. 9b is a view showing an attached surface of the skin of a pig after attachment of the microneedle array of comparative example 1 to the skin of the pig and then removal of the microneedle array from the skin of the pig.

FIG. 10a is a view showing a base part after attachment of the microneedle array of comparative example 2 to the skin of a pig and then removal of the microneedle array from the skin of the pig. FIG. 10b is a view showing an attached surface of the skin of a pig after attachment of the microneedle array of comparative example 2 to the skin of the pig and then removal of the microneedle array from the skin of the pig.

### DETAILED DESCRIPTION OF EXEMPLARY EMBODIMENTS

The present disclosure relates to a microneedle comprising a drug part, a separational function part that is water-soluble, and a base part that includes a photocurable resin.

Additionally, the present disclosure relates to a microneedle array comprising the microneedle of the present disclosure.

Further, the present disclosure relates to a method for manufacturing a microneedle array comprising,
placing a drug part composition in a mold and drying a solvent to form a drug part,
placing a separational function part composition on the drug part and drying the same to form a separational function part, and
applying a base part composition on the separational function part and curing the base part composition.

Hereinafter, the subject matter of the present disclosure is described specifically. Description of a microneedle and description of a microneedle array may be applied interchangeably.

### Microneedle 1 and Microneedle Array 2

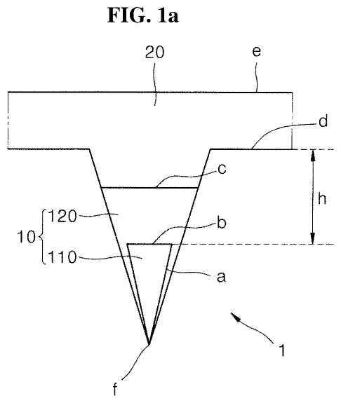

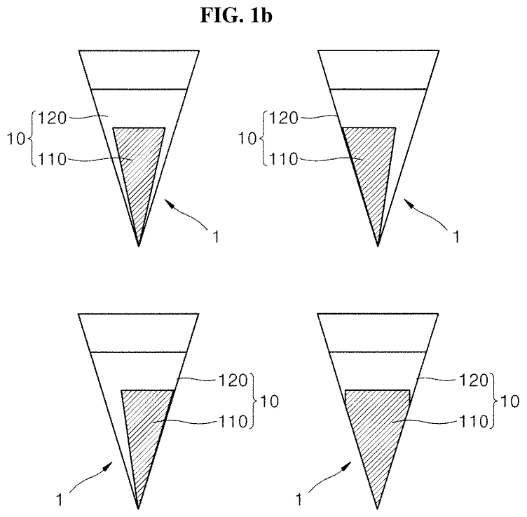

The present disclosure relates to a microneedle 1 comprising a drug part 110, a separational function part 120 that is water-soluble, and a base part 20 that includes a photocurable resin (FIGS. 1a and 1b). Additionally, the present disclosure relates to a microneedle array 2 comprising the microneedle 1 of the present disclosure.

A microneedle denotes a needle-type structure having a micrometer (μm)-unit length, and a fine structure that can penetrate the stratum corneum to ease transdermal delivery of a therapeutic agent through the skin. In the present disclosure, the multilayer microneedle denotes a microneedle comprising a drug part that includes a drug, a tip part that includes a separational function part including a water-soluble polymer, and a base part that supports the tip part.

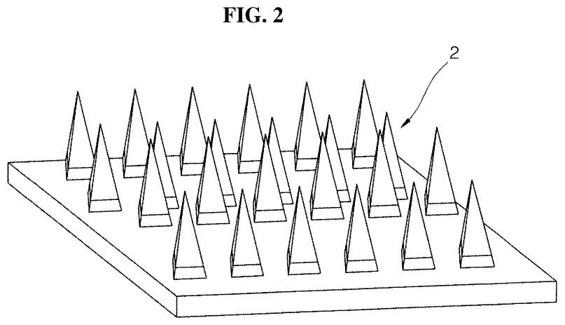

The microneedle array comprises one or more microneedles (FIG. 2). The plurality of microneedles is connected through the base part, and preferably, the base part is an integrated one. According to the present disclosure, the multilayer microneedle array may denote a microneedle array comprised of a drug part, a plurality of tip parts that includes a separational function part, and a base part that supports the plurality of tip parts.

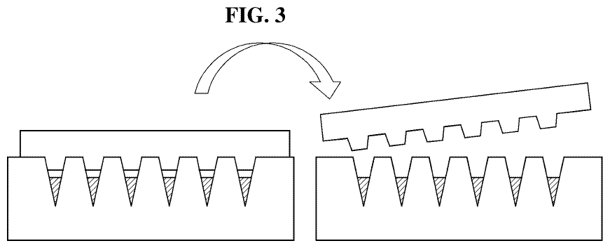

FIGS. 1a and 1b are views showing the structure of a microneedle 1 according to the present disclosure. FIGS. 1a and 1b show various embodiments of the microneedle 1 according to the present disclosure. The shapes of the drug part 110 and the separational function part 120 may vary depending on the composition of the drug part 110, the separational function part 120 and the like. Accordingly, the microneedle 1 may have a variety of cross sections. Hereinafter, common technical features of the microneedle 1 according to the present disclosure are described. The base part 20 does not contact the drug part 110 directly. Additionally, the base part 20 does not contact a drug directly. The base part 20 is attached to the separational function part 120 based on curing. A portion of the separational function part, farthest from the sharp end f of the tip part 10 of the microneedle, becomes an interface between the separational function part 120 and the base part 20, and is referred to as a separation function upper end c. The drug part contacts the separational function part, and a portion of the drug part farthest from the sharp end f of the tip part 10 of the microneedle becomes an interface between the separational function part 120 and the drug part 110, and is referred to as a drug part upper end b. Additionally, an interface between the drug part 110 and the separational function part 120 is referred to as a drug part lateral surface a. In the base part 20, the surface opposite to the tip part 10, is referred to as a base part upper end e. A portion of the base part 20 at the tip part side 10 contacts the tip part 10, and viewed with reference to the base part upper end e, comprises a concave part and a convex part. The convex part protrudes toward the tip part 10 of the microneedle, and contacts the tip part 10. At this time, an interface where the convex part of the base part 20 contacts the separational function part 120 may also be the separational function part upper end c. Ordinarily, the concave part d of the base part is parallel with the base part upper end e, considering costs and ease of processing, but not necessarily parallel with the base part upper end e. Obviously, one having ordinary skill in the art can determine the shape of the concave part d by using the shape of a forming mold. Preferably, in the case where a length h from the concave part d of the base part to the drug part upper end b is 200 μm or greater, the tip part penetrates the separational function part penetrates the stratum corneum and epidermis, and at least a portion of the separational function part readily reaches the dermis. More preferably, the length is 200 μm or greater and 1000 μm or less. The drug part 110 and the separational function part 120 constitute the tip part 10, and the tip part is a portion that remains after the microneedle is attached to the skin and then detached from the skin (FIG. 3). FIG. 3 shows that the tip part is separated from the base part when the microneedle array is attached to the skin and then detached from the skin. In the case of a microneedle of the present disclosure, when the microneedle is attached to the skin, the separational function part is dissolved within a few seconds to dozens of minutes. Thus, the microneedle array may be removed immediately after the microneedle is attached to the skin, ensuring ease of use. For example, as the microneedle in the present disclosure is attached to the skin, the separational function part is dissolved within 5 seconds to 30 minutes, preferably, within 5 minutes to 15 minutes.

In the case of a microneedle/microneedle array of the present disclosure, a drug is only included in the drug part, such that a predetermined amount of the drug is delivered into the skin, minimizing a loss of the drug. Additionally, a method for filling the drug part, the separational function part and the base part may be constituted in the same way, without additional processing, enabling mass production. The base part in the present disclosure is made of a photocurable material, enabling a crosslink within a short period of time, and heat is not applied, securing stability of a drug easily, and the tip part and the base part are readily combined, simplifying processing. Additionally, since the drug part and the base part constituting the microneedle array of the present disclosure array are separated within a few seconds to dozens of minutes, based on the dissolution of the separational function part comprised of a water-soluble polymer, the microneedle array does not need to be attached to the skin for a long period of time, and may be removed within a short period of time. Thus, a secondary infection, a foreign body sensation, and a pain caused by a gap between the skin and the microneedle may decrease. The base part is made of a photocurable hard material, thus can provide a sufficient force to allow the tip part to be evenly inserted into the skin, and requires no additional applicator to apply a uniform force, ensuring improvement in the uneven insertion of an existing microneedle array.

Further, according to the present disclosure, a liquid-phase photocurable material is applied to the upper portion of the tip part manufactured in advance, and light source is irradiated for a short period of time such as a few minutes to dozens of minutes, such that the base part is formed immediately, and at the same time, the tip part and the base part join, ensuring ease of processing and efficiency of a high yield. [54]

### Tip Part 10

The tip part comprises a drug part and a separational function part. The length of the tip part may be adjusted by adjusting the content of a solvent and a solute, or by varying an amount of a composition filling the forming mold at a time of manufacturing of a drug part composition or a separational function part composition. The microneedle needs to have a length of at least 500 μm or greater to pass through the stratum corneum of the skin, deliver a drug under the dermis and have a target amount of the drug on top of it. Further, the length of the microneedle needs to be the range between 2000 μm, preferably, the range of 750 to 1500 μm, not to damage blood vessels and nerves.

In the case of a microneedle of the present disclosure, the base part and the tip part are not separated while the microneedle passes through the stratum corneum, and the strength of the tip part is preferably 0.05 N or greater to penetrate the skin.

The multilayer microneedle of the present disclosure comprises a drug part that is inserted into the skin and dissolved or biodegraded. The tip part comprising the drug part has a sharp end, to penetrate the skin. The tip part comprises a drug part that includes a drug, and a separational function part that is comprised of a water-soluble polymer.

In one embodiment, the tip part has a pyramid shape as illustrated, for example, but the shape of the tip part is not limited. Additionally, the number of the tip parts of the microneedle array is not limited to that of the illustrated embodiment. Further, the tip part and the base part of the microneedle are comprised of multiple layers that are seperable. The microneedle may comprise an additional layer in addition to the tip part and the base part, and the tip part may comprise an additional layer in addition to the drug part and the separational function part.

### Drug Part 110

The drug part comprises a drug and a polymer. The polymer included in the drug part is a biocompatible polymer. The biocompatible polymer may be a polymer material that is degraded on its own in the body or is water-soluble, may be included in the drug part, in the form of a composition where a drug or other active ingredients are mounted on a biodegradable or water-soluble polymer material, and may include the biocompatible polymer or a composition comprising a biocompatible polymer solidified in the form of a microneedle. The polymer may be a biocompatible polymer. The water-soluble polymer of the biocompatible polymer used for the drug part may comprise hyaluronic acid or a salt thereof, carboxymethyl celluose, alginic acid, chitosan, guar gum, locust bean gum, trehalose, glucose, maltose, lactose, lactulose, fructose, turanose, melitose, melezitose, dextran, sorbitol, xylitol, pallatinite, mannitol, hydroxypropyl methyl cellulose (HPMC), ethyl cellulose, hydroxyethyl cellulose, polyalcohol, gum arabic, dextrin, starch, hydroxypropyl cellulose, cyclodextrin, pectin, carrageenan, dextran sulfate, chondroitin sulfate, polylysine, collagen, gelatin, carboxymethyl chitin, fibroin, agarose, pullulan, polyvinylpyrrolidone (PVP), polyethylene glycol (PEG) and the like. The biodegradable polymer may be one or more selected from polymethacrylate, polylactic acid (PLA), poly(glycolic acid (PGA), poly(lactic-co-glycolic acid) (PLGA), polyanhydride, polyorthoester, polyetherester, polycaprolactone, polyesteramide, poly(3-hydroxybutyric acid), poly(3-hydroxybutyrate-co-3-hydroxyvalerate) (PHBV), polyurethane, polyacrylate, ethylene vinyl acetate copolymer, cellulose acetate co-acrylate, non-degradable polyurethane, polystyrene, polyvinyl chloride, polyvinyl fluoride, polyvinylimidazole, chlorosulphonate polyolefins and the like, but not limited.

One sort or two or more sorts of the polymers may be selected to enhance mechanical strength, adjust a release rate of a drug, at which a drug is rapidly dissolved or slowly biodegradable in the body, and the like, and improve stability of a drug and the like.

In one embodiment, the polymers may be used in an immediate release form that are collapsed immediately after the polymers are directly mixed with a drug and inserted into the skin, and releases the drug within a few seconds to a few hours. In another embodiment, the polymers may be used in a sustained release form that is biodegradable for a few days to a few months after the polymers are directly mixed with a drug and inserted into the skin, and releases the drug slowly. In one embodiment, the polymer and the drug are atomized, or encapsulated to form a structure, and the structure is mixed with the polymer to manufacture a drug part. The microneedle array manufactured as described above may be used in a sustained release form where the polymer forming the structure of the drug part of the microneedle array is collapsed first after the microneedle array is inserted into the skin, while the polymers atomized or capsulated together with the drug are biodegradable in the body for a few days to a few months and release the drug slowly. The biocompatible polymer used for the drug part may be a water-soluble polymer, a water-insoluble polymer or a biodegradable polymer, and as described above, the sort of a polymer may be properly selected by one skilled in the art, depending on the sort of a drug. For example, in the case of delivery of an immediate release drug, a water-soluble polymer may be used, and in the case of delivery of a sustained release drug, a biodegradable polymer may be used.

The drug may comprise DNA, RNA, protein, peptide drugs, hormones, hormone analogues, enzymes, enzyme inhibitors, signal transfer protein or a portion thereof, an antibody or a portion thereof, a single chain antibody, binding protein or a binding domain thereof, an antigen, adherence protein, structural protein, regulatory protein, toxic protein, cytokine, a transcriptional regulatory factor, a blood clotting factor, and a vaccine. It may comprise at least any one of cyclosporine, insulins, vitamins, insulinlike growth factor-1 (IGF-1), growth hormone, sex hormones, growth hormone releasing hormone-II (GHRH-II) of peptides and the like, gonadorelin, goserelin, histrelin, leuprorelin, lypressin, octreotide, oxytocin, pitressin, secretin, sincalide, terlipressin, thymopentin, thymosine, triptorelin, bivalirudin, carbetocin, exedine, lanreotide, luteinizing hormonereleasing hormone (LHRH), nafarelin, pramlintide, T20 (enfuvirtide), thymalfasin ziconotide, interferon-α, interferon-β for multiple sclerosis, erythropoietin, follitropin beta, follitropin alpha, granulocyte colony-stimulating factor, granulocyte-macrophage colony-stimulating factor (GM-CSF), human chorionic gonadotropin, leutinizing hormones, salmon calcitonin, glucagon, Gonadotropin-releasing hormone (GNRH) antagonist, filgrastim, heparin, low molecular weight heparin and somatropin or may be one or more selected from interferon gamma, interleukin-1 alpha and beta, interleukin-3 (interleukin-1), interleukin-4, interleukin-6, interleukin-2, interleukin-17, interleukin-21, epidermal growth factors (EGFs), adrenocorticotropic hormone (ACT), tumor necrosis factor (TNF), atobisban, buserelin, cetrorelix, deslorelin, desmopressin, dynorphin A)(1-13), elcatonin, eleidosin, eptifibatide, parathyroid hormone (PTH) and the like, but is not limited. In one embodiment, the drug may be a synthetic drug, and for example, one or more selected from methotrexate, bisphosphonates, tofacitinib, acyclovir, penciclovir, naratriptan, zolmitriptan, midodrine, tizanidine, fluticasone, salmeterol, ipratropium, tacrolimus, coenzyme Q10, chitosan, botox, hydroxy acid, tetracycline, oxytetracycline, clindamycin, doxycycline, minocycline, benzocaine, mepivacaine, lidocaine, prilocaine, bupivacaine, etidocaine, articaine, procaine, propoxycaine, tetracaine, ropivacaine, butacaine, piperocaine, cocaine, chloroprocaine, proparacaine and dyclonine, benzoyl peroxide and the like, but not limited.

In one embodiment, the drug part may comprise 50 wt % or greater of a biocompatible polymer. In one embodiment, the drug part may comprise 50 wt % or less of a drug. The drug part may further comprise a proper additive to improve physical properties and the like, in addition to the biocompatible polymer and the drug.

### Separational Function Part 120

The separational function part comprises a water-soluble polymer and is dissolved within a few seconds to dozens of minutes after being inserted into the skin, such that at least one or more of tip parts and base parts are separated. The separational function part fills the forming mold secondarily, following the drug part. The separational function part has to be rapidly dissolved by the body fluid as the separational function part is inserted into the skin, such that the tip part, specifically, the drug part and the base part separate. To this end, a water-soluble biocompatible polymer highly dissoluble by the body fluid has to be used. A proper material for the water-soluble biocompatible polymer may be one or more selected from hyaluronic acid or a salt thereof, carboxymethyl celluose, alginic acid, chitosan, guar gum, locust bean gum, trehalose, glucose, maltose, lactose, lactulose, fructose, turanose, melitose, melezitose, dextran, sorbitol, xylitol, pallatinite, mannitol, hydroxypropyl methyl cellulose (HPMC), ethyl cellulose, hydroxyethyl cellulose, polyalcohol, gum arabic, dextrin, starch, hydroxypropyl cellulose, cyclodextrin, pectin, carrageenan, dextran sulfate, chondroitin sulfate, polylysine, collagen, gelatin, carboxymethyl chitin, fibroin, agarose, pullulan, polyvinylpyrrolidone (PVP), polyethylene glycol (PEG), or a mixture of two or more of them, but not limited.

A water-insoluble polymer or a poorly water-soluble polymer is hardly melted and collapsed by the body fluid within a short period of time, is hardly separated from the base part, and is not suitable for a main ingredient of the separational function part of the microneedle in the present disclosure. For example, an ingredient unsuitable for constituting the separational function part comprises polylactic acid (PLA), poly(glycolic acid) (PGA), poly(lactic-co-glycolic acid (PLGA), polyanhydride, polyorthoester, polyetherester, polycaprolactone, polyesteramide, poly(3-hydroxybutyric acid), Poly(3-hydroxybutyrate-co-3-hydroxyvalerate (PHBV), polyurethane, polyacrylate, ethylene vinyl acetate copolymer, cellulose acetate co-acrylate, non-degradable polyurethane, polystyrene, polyvinyl chloride, polyvinyl fluoride, polyvinylimidazole, chlorosulphonate polyolefins and the like.

The separational function part of the present disclosure is water-soluble, while the base part is water insoluble. Since the separational function part and the base part have a different physical property, a drug is not diffused, and an accurate dose of a drug is provided to the skin.

The separational function part is rapidly dissolved by the body fluid as the separational function part is inserted into the skin, and separates the drug part and the base part. To this end, the separational function part needs to reach the dermis of the skin abundant with moisture and body fluids by passing through the stratum corneum and the epidermis of the skin. Accordingly, the length of the microneedle, at which the microneedle is inserted from the surface of the skin to the inside of the skin, has to be 200 μm or greater, and a portion of the separational function part has to reach the dermis of the skin at a time when the separational function part is inserted into the skin.

The separational function part may comprise 60 wt % to 100 wt % of a water-soluble biocompatible polymer. The separational function part may further comprise a proper additive to improve physical properties such as a separation speed and the like, in addition to the water-soluble biocompatible polymer.

### Base Part 20

The base part is a planar layer to which the microneedle is attached, is a support body that supports the tip parts of at least one or more microneedles, and preferably, comprises a photocurable material but is not limited. The base part in the present disclosure ensures improvement in the uneven insertion of an existing microneedle array, and uses a base part of a photocurable material to apply a uniform force to the entire tip part without an additional applicator, such that the entire tip part may penetrate the skin. The base part of the present disclosure may comprise a photocurable material, preferably, a photocurable resin. The photocurable material is a material that is formed by initiating a initiation reaction within a short period of time with a radical or a cation generated from a photo-initiator by light energy of ultra violet (UV), a light emitted diode (LED), visible light and the like and curing a reactive monomer or oligomer through a consecutive reaction of photo polymerization, photo crosslink and the like. The photocurable material may be used in a variety of fields such as medicine, electricity, optics, aerospace, vehicles, home appliances, metal working, alternative energy and the like. A photocurable mixture may support a plurality of tip parts, have enough mechanical strength, to withstand while the tip part penetrates the skin, and have flexibility to be attached to a curved skin surface flexibly. Additionally, the photocurable mixture used as the base part composition in the present disclosure uses a biocompatible material, preferably, since the photocurable mixture is attached to the skin directly and inserted into the body.

The base part composition may be a photocurable mixture. The photocurable mixture comprises a photocurable monomer, a photocurable oligomer, and a photoinitiator, and further comprises a supplement selectively.

The photocurable monomer may comprise 2-ethylhexyl acrylate, 2-hydroxyethyl acrylate), 2-hydroxypropyl acrylate and the like as a monofucntional monomer, and comprise 1,3-butanediol diacrylate, 1,4-butanediol diacrylate, 1,6-hexanediol diacrylate, diethylene glycol diacrylate, tripropyleneglycol diacrylate, neopentylgylcol diacrylate, and polyethyleneglycol 400 diacrylate as a difunctional monomer. Additionally, as a multifunctional monomer, trimethylolpropane triacrylate, dipentaerythritol hexaacrylate and the like can be used.

The photocurable oligomer may comprise epoxy acrylate, urethane acrylate, polyester acrylate, polyether acrylate, unsaturated acryls, silicon acrylate and the like as acrylates. Unsaturated polyesters may be used as the photocurable oligomer but have a slow curing speed. However, unsaturated polyesters are not preferred considering the productivity of the microneedle array. Further, a positive-ion oligomer may comprise cycloaliphatic epoxy, glycidyl ether epoxy and the like.

As a photoinitiator, there are α-hydroxy ketones, α-amino ketons, benzylmethyl ketal (benzyldimethyl ketal (BDK)) and the like. The photoinitiator may comprise phenyl glyoxylates, acryl phosphine oxides, oxime esters, benzoin ether, benzyl ketal, α-dialkoxyacetophenone, α-hydroxy alkylphenone, α-amino alkylphenone, acylphosphine oxide, benzophenone/amine, thioxanthon/amins and the like, and among them, may comprise one or more selected from 1-hydroxycyclohexylphenylketone, bis(2,4,6-trimethylbenzoyl)-phenylphosphine oxide, 2-methyl-1-[4-(methylthio)phenyl]-2-morpholino-porpane-1-one, 2,4-diethylthioxanothone), camphorquinone and the like.

At a time when the base part composition is applied and photocured, at least any one or more of ultraviolet (UV) light, visible light, LED light, and UV-LED light may be used as a light source, depending on the base part composition, i.e., the composition and sort of a photocurable mixture, but the light source is not limited.

The hardness of the base part is preferably Shore A 50 to 100, or Shore D 40 to 85, based on the Shore hardness scale.

### Method for Manufacturing Microneedle Array

The present disclosure relates to a method for manufacturing a microneedle comprising: placing a drug part composition in a mold and drying the same to form a drug part; placing a separational function part composition on the drug part and drying the same to form a separational function part; and applying a base part composition on the separational function part and curing the same.

Also, the present disclosure relates to a method for manufacturing a microneedle array comprising: placing a drug part composition in a mold and drying the same to form a drug part; placing a separational function part composition on the drug part and drying the same to form a separational function part; and applying a base part composition on the separational function part and curing the same. The mold may comprise a plurality of grooves into which the drug part composition is put.

The drying step involves removing a solvent at least partially or completely, and a drying method is not limited. For example, the drying step in the present disclosure may be performed based on evaporation, volatilization, vaporization and the like of a solvent.

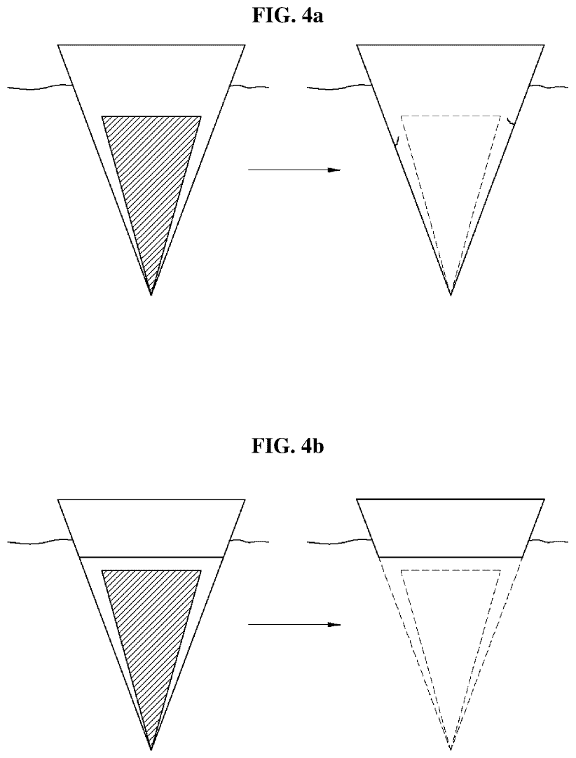

At a time of manufacturing a double layer microneedle array, a mold is filled with a drug part composition, and then a solvent is dried, resulting in vaporization of the solvent and shrinkage of a drug part. At this time, there is a gap between the mold and the drug part, and in the case where a photocurable polymer constituting the base part is applied, the photocurable polymer infiltrates into the gap. At this time, a boundary between the tip part comprising a drug and the base part is blurred, and the separability of the tip part deteriorates, making it hard to deliver an accurate dose of the drug. Additionally, an insoluble hetero polymer material infiltrates into the skin and puts pressure in the skin, causing a pain, during attachment of a patch. In the case where a photo-crosslinked polymer used for the base part infiltrates up to the tip part, there is a problem that it may remain not dissolved or broken after use, causing a foreign body sensation or a pain (FIG. 4a shows that an existing double layer microneedle is attached to the skin such that the drug part is dissolved). However, in the case of a multilayer microneedle of the present disclosure, since the drug part and the base part do not contact each other directly, and the separational function part directly contacting the base part is dissolved, it is less likely that the base part is broken or remains in the skin, at a time when the drug part is absorbed into the body after attachment to the skin (FIG. 4b).

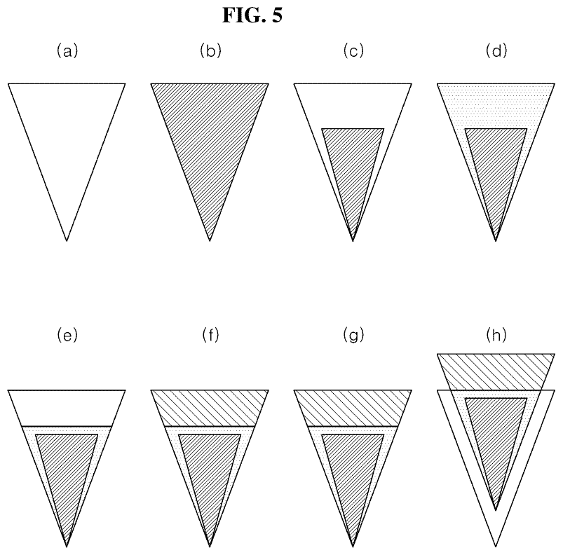

In the method for manufacturing a microneedle or a microneedle array of the present disclosure, the drug part is formed, and then a water-soluble polymer fills the separational function part secondarily and is dried, to fill a gap between a forming mold and the drug part formed, thereby solving the above problem. That is, in the method for manufacturing a microneedle array of the present disclosure, a forming mold is prepared ((b) in FIG. 5), and then filled with the drug part composition ((b) in FIG. 5), a solvent of the drug part composition is dried to form the drug part ((c) in FIG. 5), and then the separational function part composition is filled ((d) in FIG. 5), and a solvent of the separational function part composition is dried to form the separational function part ((e) in FIG. 5). Additionally, the base part composition is applied onto the separational function part ((f) in FIG. 5), cured to form the microneedle ((g) in FIG. 5) and separated from the forming mold ((h) in FIG. 5).

In the method for manufacturing a multilayer microneedle and/or a multilayer microneedle array of the present disclosure, filling the forming mold with a composition may be properly selected from filling the mold with a solution and applying pressure, using centrifugation, filling in the tip part one by one using a microjet or a precise filling machine, filling a solution and removing bubbles under vacuum conditions, and the like, but not limited.

Drying of a solvent may adopt a variety of existing methods such as hot-air drying, freeze drying, vacuum drying and the like properly. One skilled in the art may select and use an existing method of drying a solvent properly, as long as the stability of the drug of the tip part is ensured, mechanical strength does not deteriorate, or deformation in the step of drying a solvent is not caused, and the drying method is not limited.

### Step of Forming Drug Part

The method for manufacturing a microneedle, and the method for manufacturing a microneedle array, in the present disclosure, comprise placing the drug part composition into the mold and drying the same to form the drug part. At this time, the drug part composition primarily fills an engraving forming mold prepared, and in the drying process, a solvent is removed partially or completely to form the drug part.

When the drug part composition is placed into the mold, the volume of the drug part composition is greater than the volume of the drug part. In the present disclosure, even if the volume of the drug part decreases due to drying by solvent volatilization and the like, the separational function part is placed between the drug part and the base part, thereby preventing the base part composition from infiltrating into the drug part or contact between the drug of the drug part and the base part.

### Step of Forming Separational Function Part

The method for manufacturing a microneedle, and the method for manufacturing a microneedle array, in the present disclosure, comprise placing the separational function part composition on the drug part and drying the same, to form the separational function part. The separational function part composition made of a water-soluble polymer is secondarily filled in the drug part formed in the primary filling and solvent drying and then dried, and the separational function part is formed, to form a final tip part. At this time, it is not significantly limited if the stability of the drug contained in the tip part may be ensured and unless the process does cause deformation during solvent removal or lowering of mechanical strength. The separational function part composition is water-soluble, and the separational function part manufactured as described above is also water-soluble.

### Step of Forming Base Part

The method for manufacturing a microneedle, and the method for manufacturing a microneedle array, in the present disclosure, comprise applying the base part composition on the separational function part and curing the same. The base part composition is a photocurable resin composition, and the base part may be formed by filling and applying it and curing the photocurable resin composition by irradiating a light source.

### Step of Separating Microneedle Array

The method for manufacturing a microneedle, and the method for manufacturing a microneedle array, in the present disclosure, may further comprise separating the microneedle array from the forming mole after the base part is formed.

Advantages and features in the present disclosure and a method of ensuring the same can be clearly understood from embodiments that are described hereinafter. However, embodiments are not limited to the embodiments set forth herein, and can be modified and changed in various different forms. The embodiments in the disclosure are provided such that the disclosure can be through and complete and fully convey its scope to one having ordinary skill in the art. The scope of protection of the subject matter is to be defined according to the following claims.

### Embodiment 1

In embodiment 1, purified water was added to 50 g of hyaluronic acid and Brilliant blue FCF 0.025 g, to make 100 g, and stirred sufficiently for 60 minutes at room temperature, to manufacture a drug part composition. The drug part composition was defoamed for 10 minutes at 750 mmHg at room temperature, and filled an engraving microneedle array forming mold of a pyramid shape having a depth of 1200 μm, with a 30-gauge needle, and a solvent was removed for 3 hours at room temperature, to form a drug part.

Additionally, 50 g of purified water was put into 50 g of hyaluronic acid and 0.025 g of Congo red, stirred for 60 minutes at room temperature, and defoamed for 10 minutes at 750 mmHg at room temperature, to manufacture a separational function part composition that is water-soluble.

The separational function part composition was placed on the forming mold where the drug part was formed, with a 30-gauge needle, and was dried for 5 hours at room temperature, to manufacture a tip part of a microneedle array finally.

A photocurable mixture (35 wt % of glycerol dimethacrylate, 62 wt % of urethane dimethacrylate as an oligomer, 3 wt % of bis (2,4,6-trimethyl benzoyl)-phenylphosphine oxide) as a photoinitiator) was applied onto the forming mold where the tip part was formed, and irradiated with UV-LED light and cured, and then separated from the forming mold, to manufacture a microneedle array.

### Embodiment 2

In embodiment 2, 1 g of poly (lactic-co-glycolic acid) (PLGA) was melted in 20 g of dichloromethane, 0.025 g of Congo red was melted in 0.5 g of acetone, and stirred sufficiently for 60 minutes at room temperature, to manufacture a drug part composition. The drug part composition was defoamed for 10 minutes at 750 mmHg at room temperature, and filled an engraving microneedle array forming mold of a pyramid shape having a depth of 1200 μm, with a 30-gauge needle, and a solvent was removed for 12 hours at 60° C., to form a drug part.

Additionally, 50 g of purified water was put into 50 g of hyaluronic acid and 0.025 g of Brilliant blue FCF, stirred for 60 minutes at room temperature, and defoamed for 10 minutes at 750 mmHg at room temperature, to manufacture a separational function part composition that is water-soluble.

The separational function part composition was placed on the forming mold where the drug part was formed, with a 30-gauge needle, and was dried for 5 hours at room temperature, to manufacture a tip part of a microneedle array finally.

### Manufacturing Example 1

A drug part composition was manufactured, and a drug part was formed, in the same way that embodiment 1 was manufactured.

Additionally, 50 g of carboxymethyl cellulose was put into 50 g of purified water, stirred for 60 minutes at room temperature, defoamed for 10 minutes at 750 mmHg at room temperature, to manufacture a separational function part composition that is water-soluble.

A microneedle array was manufactured in the same way that embodiment 1 was manufactured, except that a separational function part was formed by the separational function part composition.

### Manufacturing Example 2

A drug part composition was manufactured, and a drug part was formed, in the same way that embodiment 1 was manufactured.

Additionally, 40 g of hyaluronic acid, 10 g of trehalose, and 50 g of purified water were stirred for 60 minutes at room temperature, and defoamed for 10 minutes at 750 mmHg at room temperature, to manufacture a separational function part composition that is water-soluble.

A microneedle array was manufactured in the same way that embodiment 1 was manufactured, except that a separational function part was formed by the separational function part composition.

### Manufacturing Example 3

A microneedle array was manufactured in the same way that embodiment 1 was manufactured, except that a separational function part was manufactured by using hydroxypropylmethylcellulose rather than hyaluronic acid.

### Manufacturing Example 4

A microneedle array was manufactured in the same way that embodiment 1 was manufactured, except that L-ascorbic acid was used instead of Brilliant blue FCF.

### Comparative Example 1

In comparative example 1, purified water was added to 50 g of hyaluronic acid and Brilliant blue FCF 0.025 g, to make 100 g, and stirred sufficiently for 60 minutes at room temperature, to manufacture a drug part composition. The drug part composition was defoamed for 10 minutes at 750 mmHg at room temperature, and then filled an engraving microneedle array forming mold of a pyramid shape having a depth of 1200 μm, with a 30-gauge needle, and a solvent was removed for 3 hours at room temperature, to form a drug part.

A photocurable mixture (35 wt % of glycerol dimethacrylate, 62 wt % of urethane dimethacrylate as an oligomer, 3 wt % of bis (2,4,6-trimethyl benzoyl)-phenylphosphine oxide) as a photoinitiator) was applied onto the forming mold where the drug part was formed, and irradiated with UV-LED light and cured, and then separated from the forming mold, to manufacture a microneedle array.

### Comparative Example 2

A drug part composition was manufactured like embodiment 2, and a drug part was formed in the same way that embodiment 2 was manufactured.

A photocurable mixture (35 wt % of glycerol dimethacrylate, 62 wt % of urethane dimethacrylate as an oligomer, 3 wt % of bis (2,4,6-trimethyl benzoyl)-phenylphosphine oxide) as a photoinitiator) was applied onto the forming mold where the drug part was formed, and irradiated with UV-LED light and cured, and then separated from the forming mold, to manufacture a microneedle array.

### Experimental Example 1

The microneedle arrays of embodiments 1 and 2, and comparative examples 1 and 2 were observed with a microscope.

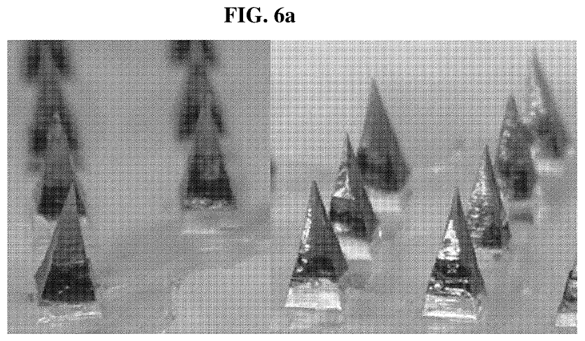

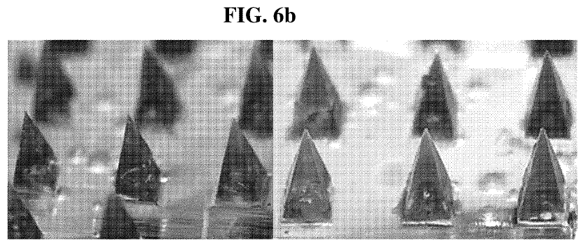

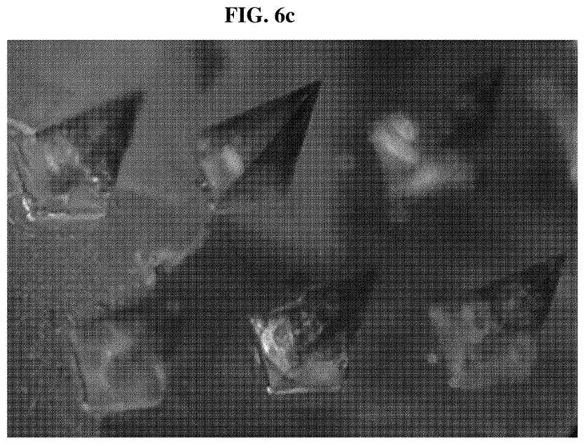

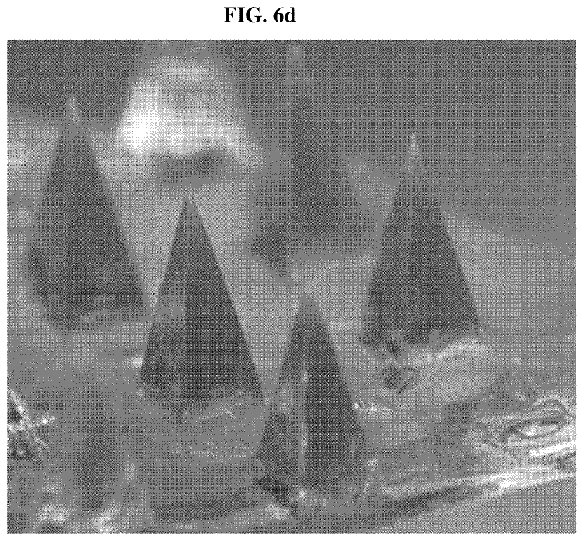

As a result, it turned out that the base part and the drug part do not directly contact each other, in the microneedle arrays of embodiments 1 and 2. However, it turned out that the base part and the drug part contacted each other directly and that the photocurable mixture forming the base part infiltrated into the drug part, in the microneedle arrays of comparative examples 1 to 2 (FIG. 6a: embodiment 1, FIG. 6b: embodiment 2, FIG. 6c: comparative example 1, FIG. 6d: comparative example 2).

### Experimental Example 2

The microneedle arrays of embodiments 1 and 2, and comparative examples 1 to 2 were pressed for 10 seconds to be inserted to pig skin and then detached from the pig skin. Then the degree of separation between the base part and the tip part was confirmed by observation with a microscope.

As a result, in terms of the microneedle arrays of embodiments 1 to 2 which were attached to the pig skin and then detached from the pig skin, it turned out that the tip part separated from the base part completely.

However, in comparative example 1, at least a portion of the tip part (at this time, the drug part) in most of the microneedles was still attached to the base part. Thus, it turned out that the separation occurred at a position where both the tip part and the base part were present or at the tip part. In comparative example 2, it turned out that the tip part was still attached to the base part in most of the microneedles. Thus, the addition of a water-soluble polymer to the separational function part prevented the infiltration of the base part into the tip part, which occurred in the microneedle array of comparative examples 1 to 2, and improved the separability of the tip part and the base part.

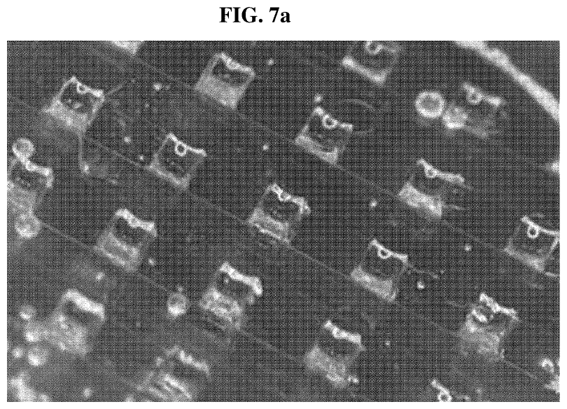

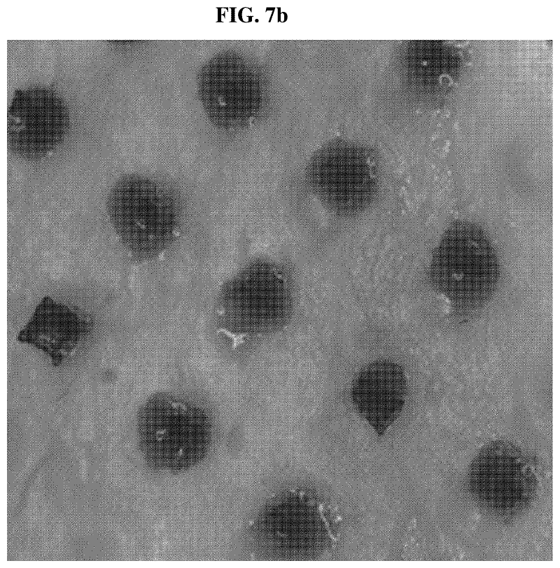

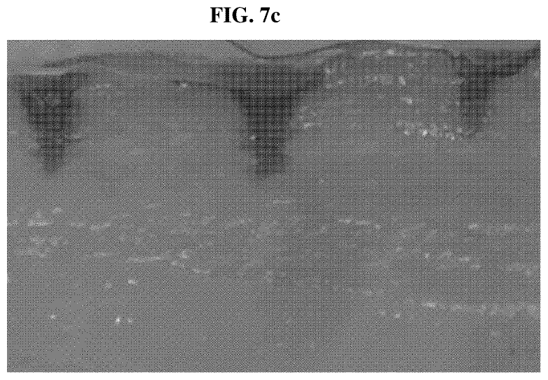

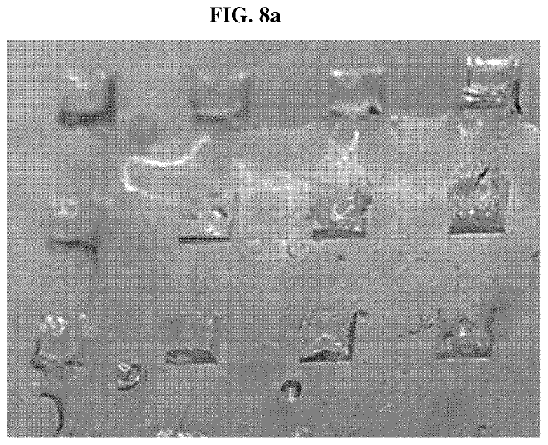

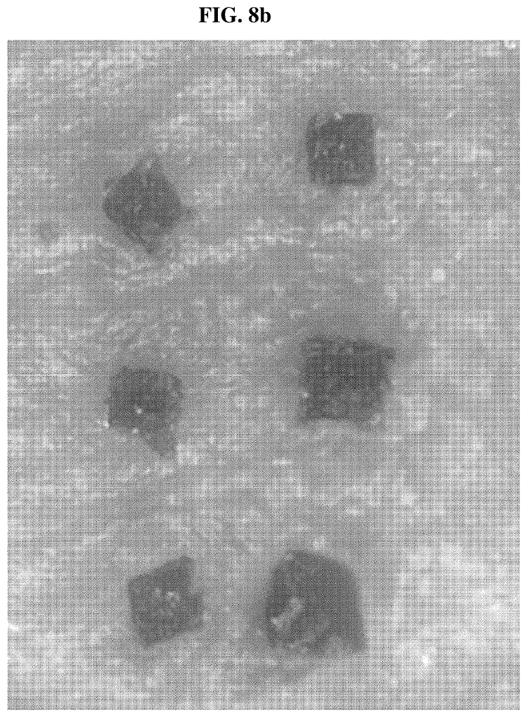

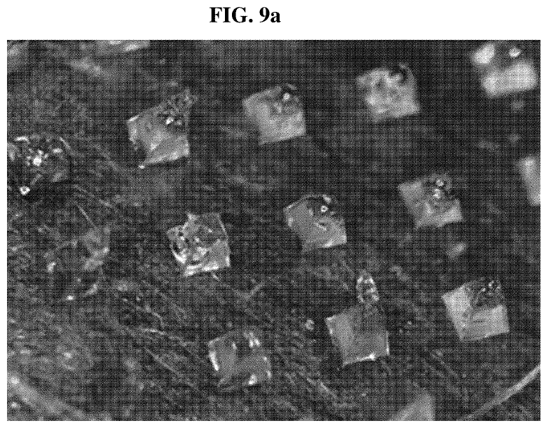

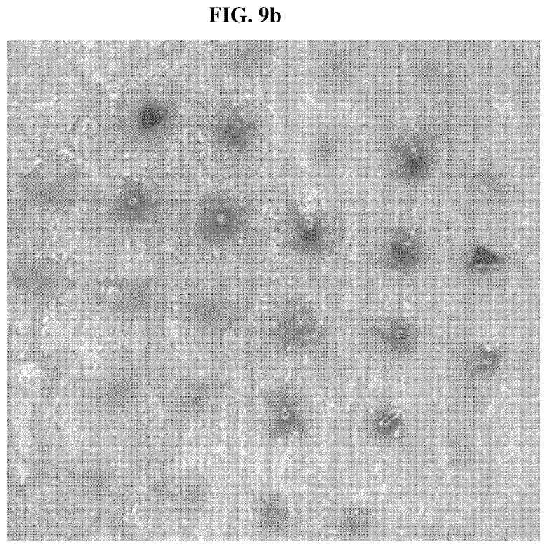

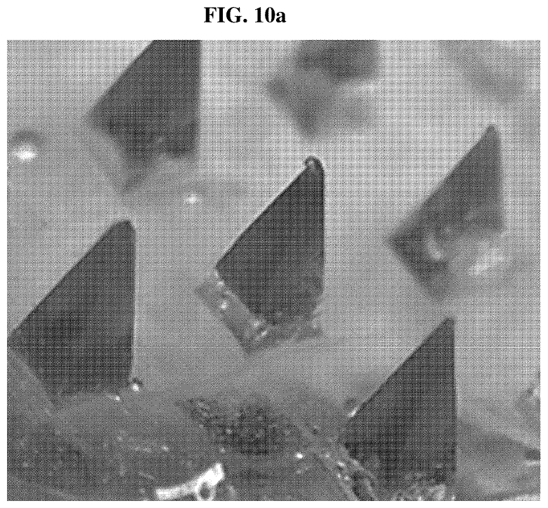

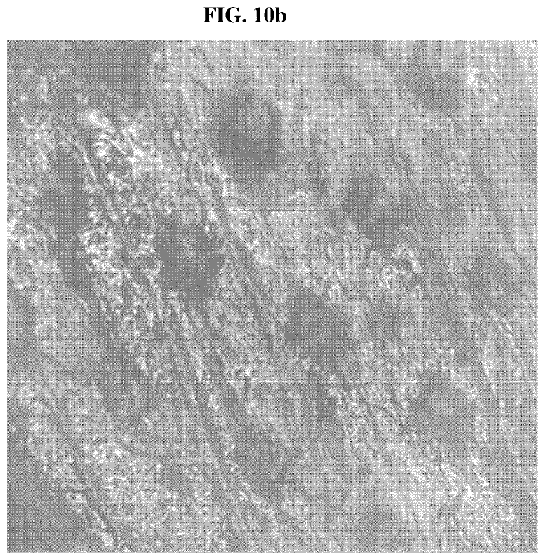

Further, the pig skin was observed after the microneedle array was attached to the pig skin and then detached from the pig skin. As a result, when the attached surface was observed after the attachment of the microneedle array of embodiments 1 to 2 to the pig skin, it turned out that the tip part separated from the base part and passed through the skin, in the microneedle array. It was revealed that the microneedle array of embodiments 1 to 2 has enough mechanical strength to pass through the stratum corneum of the skin and delivers an accurate dose of a drug into the skin. The microneedle array of comparative example 1 also passed through the stratum corneum of the skin, but in most cases, a portion of the base part was found in the pig skin, or the drug part was removed together with the base part. The tip part of the microneedle array of comparative example 2 passed through the stratum corneum of the skin, but was removed together with the base part at a time of removal. Thus, the addition of the separational function part prevented the infiltration of the base part into the tip part, which occurred in the double layer microneedle array of comparative examples 1 to 2, and improved the separability of the tip part and the base part. (FIG. 7a: a view showing a base part after attachment of the microneedle array of embodiment 1 to the skin of a pig and then removal of the microneedle array from the skin of the pig; FIGS. 7a and 7c: views showing an attached surface of the skin of a pig after attachment of the microneedle array of embodiment 1 to the skin of the pig and then removal of the microneedle array from the skin of the pig; FIG. 8a: a view showing a base part after attachment of the microneedle array of embodiment 2 to the skin of a pig and then removal of the microneedle array from the skin of the pig; FIG. 8b: a view showing an attached surface of the skin of a pig after attachment of the microneedle array of embodiment 2 to the skin of the pig and then removal of the microneedle array from the skin of the pig; FIG. 9a: a view showing a base part after attachment of the microneedle array of comparative example 1 to the skin of a pig and then removal of the microneedle array from the skin of the pig; FIG. 9b: a view showing an attached surface of the skin of a pig after attachment of the microneedle array of comparative example 1 to the skin of the pig and then removal of the microneedle array from the skin of the pig; FIG. 10a: a view showing a base part after attachment of the microneedle array of comparative example 2 to the skin of a pig and then removal of the microneedle array from the skin of the pig; FIG. 10b: a view showing an attached surface of the skin of a pig after attachment of the microneedle array of comparative example 2 to the skin of the pig and then removal of the microneedle array from the skin of the pig).

1-11. (canceled)

12. A method for manufacturing a microneedle array, comprising:
placing a drug part composition in a mold and drying a solvent, to form a drug part;
placing a separational function part composition which is water-soluble on the drug part and drying the same, to form a separational function part; and
applying a base part composition which is a photocurable resin composition on the separational function part and curing the same,
wherein a length from a concave part of the base part to an upper end of the drug part is 200 m or greater, and
wherein the microneedle array comprises plurality of microneedles, and the microneedle array is a microneedle array whose separational function part is dissolved within 5 seconds to 30 minutes when attached to the skin.

13. The method of claim 12, wherein the step of forming a drug part comprises placing a drug part composition in the mold so that the volume of the drug part composition is greater than the volume of the drug part.

14. The method of claim 12, wherein the step of forming the separational function part comprises filling a gap between the mold and the drug part with the separational function part composition.

15. The method of claim 12, wherein the microneedle comprises a drug part, a separational function part that is water-soluble and in contact with the drug portion, and a base part that comprises a photocurable resin and is in contact with the separational function part.

16. The method of claim 15, wherein the microneedle array is a microneedle array in which the base part is attached to the separational function part based on curing, the portion of the base part toward the separational function part comprises a concave part and a convex part, the convex part of the base part contacts the separational function part directly, the base part and the separational function part are made of different materials, the separational function part of the microneedle does not directly contact the separational function part of other microneedle, and the microneedles are connected by the base part.
